(19) United States
(12) Patent Application Publication
(10) Pub. No.: US 2003/0228580 A1
(43) Pub. Date: Dec. 11, 2003

(54) Method for enhancing DNA base-calling accuracy

(76) Inventors: ChangSheng Liu, State College, PA (US); Jiang Rao, Beijing (CN)

Correspondence Address: PENNIE & EDMONDS LLP, 1667 K STREET NW, SUITE 1000, WASHINGTON, DC, 20006

(21) Appl. No.: 10/163,994

(22) Filed: Jun. 7, 2002

(51) Int. Cl. C12Q 1/68; G06F 19/00; G01N 33/48; G01N 33/50

(52) U.S. Cl. 435/6; 702/20; 204/461

(57) ABSTRACT

One embodiment of the present invention relates to a method of treating data derived from a separation of a nucleic acid sample. The data, which include features indicating the presence of different nucleic acid fragments in the sample are transformed on the basis of spacings between the features to obtain transformed data having a substantially constant spacing between features. Another embodiment of the present invention relates to an electrophoresis separations apparatus having at least one separation volume, a detector, and a processor. The processor is configured to transform the separations data based upon the spacing between features to obtain transformed data having a substantially constant spacing between features.

### TECHNICAL FIELD

The present invention relates to determining structure or size relationships between electrokinetically-separated components of a sample. More particularly, the present invention relates to a method and apparatus for improving the accuracy of DNA base-calling.

### BACKGROUND

Current electrophoresis systems collect time domain data having peaks that indicate the presence of separated species. Samples are typically separated in a separation volume or lane defined by one or more capillaries, micro-fabricated channels, or lanes in a slab gel. One of the most important applications of electrophoresis systems is DNA sequencing, in which the sequence of the four bases within a particular sample of DNA is determined. In four-color fluorescent sequencing, each fragment is tagged with one of four fluorescent tags. Each of the four tags preferentially binds to fragments terminating with one of four bases. The identity of the fluorescent tag and the corresponding terminal base can be determined from the wavelength range of the tag's fluorescence, which is detected as the tagged fragment migrates through a detection zone. Typically, the detection zone is defined by a focused laser beam. The relative sizes of a series of fragments can be determined from the detection order because, in the absence of errors, smaller DNA fragments migrate faster and reach the detection zone prior to larger fragments. Accordingly, the sequence of bases in a DNA molecule can be determined from the fluorescence wavelengths of the tags bound to sequentially detected fragments.

In the presence of mobility shift errors, however, the sequence of detected fragments does not correspond to the actual sequence of fragments in the sample. One type of mobility shift results from the use of four different fluorescent tags. Each of the four fluorescent tags affects the mobility of tagged fragments to a different extent. For example, one of the tags may cause tagged fragments to migrate relatively slowly so that they are overtaken by faster moving, larger fragments tagged with a different dye. Correction for such mobility shifts is complicated at least in part because the magnitude of the tag-induced migration differences is larger for smaller fragments and varies as a non-linear function of fragment size.

Four color sequencing data exhibit other non-linearities that complicate attempts to correct mobility shifts errors. For example, even in the absence of mobility differences caused by different tags, the fragment migration rate is not a linear function of fragment size. Thus, four color sequencing data exhibit several sources of non-linearity.

Available software packages attempt to correct for the mobility shift errors by analyzing only sub-sections of a complete range of separations data. The process, however, must be repeated many times using as many as 25 parameters to process all of the time domain data. After analyzing the data, these programs attempt to correct mobility shifts using the same large number of parameters. Data taken under different experimental conditions, of course, are analyzed with different parameters, and these parameters are difficult to control. Moreover, different parameters are required for each separation volume because different separation volumes, such each capillary in an array, have different physical characteristics that influence the migration behavior.

U.S. Pat. No. 5,916,747 to Gilchrist discloses alignment of electrophoresis data traces obtained from electrophoretic separations run in different lanes to account for mobility differences between the distinct lanes. Even within a single separation lane or zone, however, different portions of a DNA sample exhibit mobility differences based on such factors as physical, chemical, and electrical properties of the portions being separated. Improved methods are needed to correct for mobility differences between different portions of a DNA sample migrating within a separation zone.

### SUMMARY OF THE INVENTION

The present invention relates to a method and apparatus for treating data derived from a separation of a nucleic acid sample. The method preferably comprises obtaining separations data having features indicating the presence of different nucleic acid fragments in the sample and transforming the data based upon spacings between the features to obtain transformed data having a substantially constant spacing between features. Preferably, the features are peaks in the data.

In a preferred embodiment, the different nucleic acid fragments include DNA fragments that terminate with different bases. The DNA fragments are preferably marked or tagged with different fluorescent tags to allow the fragments terminating with different bases to be identified.

The method preferably further comprises determining a spacing in distance or time between each of the first and second members of a plurality of pairs of features. The spacing between the first and second members of each of the pairs is preferably normalized by a number of features determined from the data. Preferably, the spacing in time or distance between each pair of features is normalized by one more than the number of other features that appear in the data between the first and second members of each pair of features.

In one embodiment, a mapping function is fit to the spacings between the first and second members of the pairs of features. Preferably, the mapping function is fit to the normalized spacings. The mapping function is preferably used to map the time or distance corresponding to each feature onto a transformed time or distance.

A preferred embodiment of the present method further comprises determining a mobility difference in the transformed data between a plurality of features indicating the presence of DNA fragments terminated with a first base and a plurality of features indicating the presence of DNA fragments terminated with a second, different base. The fitting function, preferably comprising an exponential term, is fit to the mobility differences. A length of at least one fragment is determined on the basis of the fit of the fitting function to the mobility differences.

Another embodiment of the present invention relates to a separation apparatus having at least one separation volume, a detector, and a processor. The processor is preferably configured to obtain intensity-time data from the electrophoretic separation, the intensity-time data comprising features associated with bases in the DNA sample, transform the data based upon the spacing between features to obtain transformed data having a substantially constant spacing between features. In a preferred embodiment, the electrophoretic separation apparatus comprises a plurality of separation zones and the apparatus is adapted to obtain a plurality of intensity-time data associated with simultaneous separation of a plurality of DNA samples.

Yet another embodiment of the present invention relates to a method of treating data derived from a separation of a nucleic acid sample. The method preferably comprises obtaining separations data including features indicating the presence of different nucleic acid fragments in the sample and calculating a normalized spacing between each of a plurality of pairs of features by determining a spacing in time or distance between the first and second members of each pair and normalizing each spacing by a value determined from the number of other features between the first and second members of each pair. The data are preferably transformed based upon the normalized spacings to obtain transformed data having a substantially constant spacing between adjacent features.

### BRIEF DESCRIPTION OF THE DRAWINGS

The present invention is discussed below in reference to the drawings in which:

FIG. 1 shows a flow chart outlining the steps in correcting mobility shifts according to the present invention;

FIGS. 2A-C show plots of intensity-time data obtained from an electrophoretic separation of a DNA sample;

FIG. 3A shows the migration time of bases in frames as a function of base number;

FIG. 4B shows the measured spacing between bases as a function of base number;

FIGS. 4A-C shows the transformed data from FIG. 2 plotted with constant spacing between peaks;

FIGS. 5A-C shows additional transformed data from the electrophoretic separation of FIG. 2, with the data plotted with constant spacing between peaks;

FIG. 6 shows mobility curves plotted as a function of base number;

FIG. 7 shows a schematic of an embodiment of an apparatus of the invention.

### DETAILED DESCRIPTION OF THE PREFERRED EMBODIMENT

The present invention relates to a method and apparatus for correcting differences in separation mobilities or other transport properties among compounds in a sample. Preferred compounds include, for example, nucleic acids, proteins, peptides, homologous series of molecules. More preferably, the compounds comprise DNA, such as DNA fragments that are tagged to indicate a particular property, such as a termination with a particular base. Upon excitation with a suitable light source, the tag may emit electromagnetic radiation, such as fluorescence, or causes, such as by energy transfer, another compound to emit electromagnetic radiation. Tags having other properties, such as an absorption cross section, a Raman scattering cross section, or a radioactive emission may also be used.

An electrokinetic separation using a sieving matrix is preferred for separating or distinguishing the compounds. By electrokinetic, it is meant a separation relying on some combination of electro-osmotic and electrophoretic forces to drive sample components. Electrokinetic separations can be performed using, for example, capillaries, slab gels, microfabricated channels and the like. An example of a suitable electrophoretic separations apparatus is disclosed in U.S. Pat. No. 6,027,627 to Li et al., which patent is hereby incorporated to the extent necessary to understand the present invention. Other suitable separations apparatuses include the AUTOMATED MICROFLUIDICS SYSTEM 90 manufactured by Caliper Technologies Corp., Mountain View, Calif., and the ABI PRISM 7900HT system manufactured by Applied Biosystems, Foster City, Calif. The present invention may alternatively be applied to other separation techniques, such as high pressure liquid chromatography.

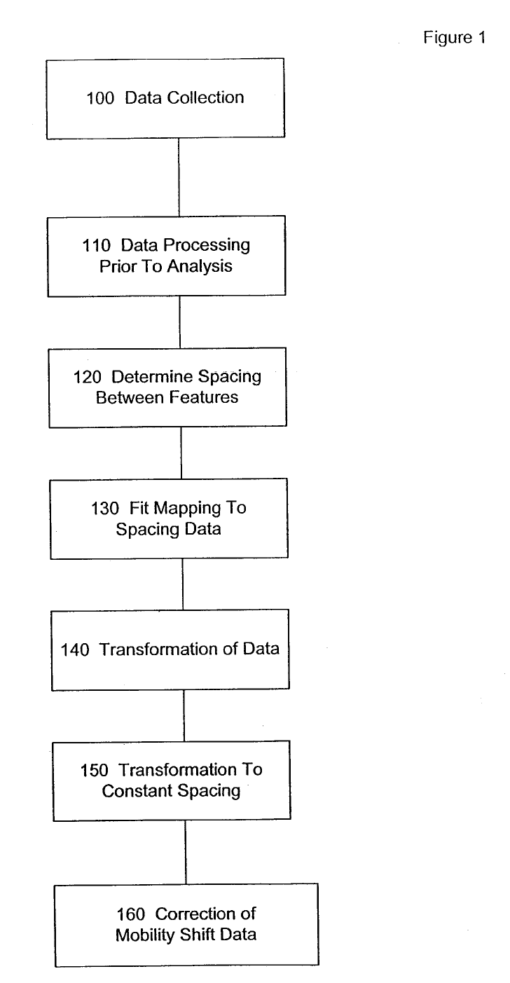

As seen in the flow chart of FIG. 1, the method of the present invention preferably includes data collection 100, in which separations data are provided or acquired;

optional pre-analysis processing 110, in which data are processed such as by smoothing or performing background correction; a determination of spacings between features 120; fitting a mapping function to the spacings data 130; and data transformation 140. Data transformation 140 may include data transformation to a constant feature spacing 150. Mobility shifts within the transformed data may be corrected 160. Steps of the invention are discussed in detail below.

100 Data Collection. As a non-limiting example to illustrate data collection for the present invention, an SCE 9610 capillary gel electrophoresis system from SpectruMedix Corp. was used to collect fluorescence-time data from a capillary electrohporetic separation of a plurality of nucleic acid fragments. A 5% copolymer in 1×TBE buffer at pH 8.4 was used as a sieving matrix in capillaries having an effective length of 55 cm (70 cm total), ID 75 um and an OD of 200 um. The capillaries were obtained from Polymicro Technologies Inc. (Phoenix, Ariz.). A voltage of −8 kV was applied to the capillary in a 96-capillary array. An electro-dynamic injection, 4 kV and 60 sec, was applied with pGEM-3Zf(+) samples. The separation was performed at 60° C. The above-mentioned separation conditions merely represent exemplary conditions suitable for separating nucleic acid fragments. It should be understood that the present invention is not limited to separations performed under a particular set of conditions. Additionally, although the method and apparatus of the invention can be applied to samples simultaneously separated in multiple capillaries, such as 96 capillaries, the present example selects a single sample separated within one capillary to demonstrate the principles.

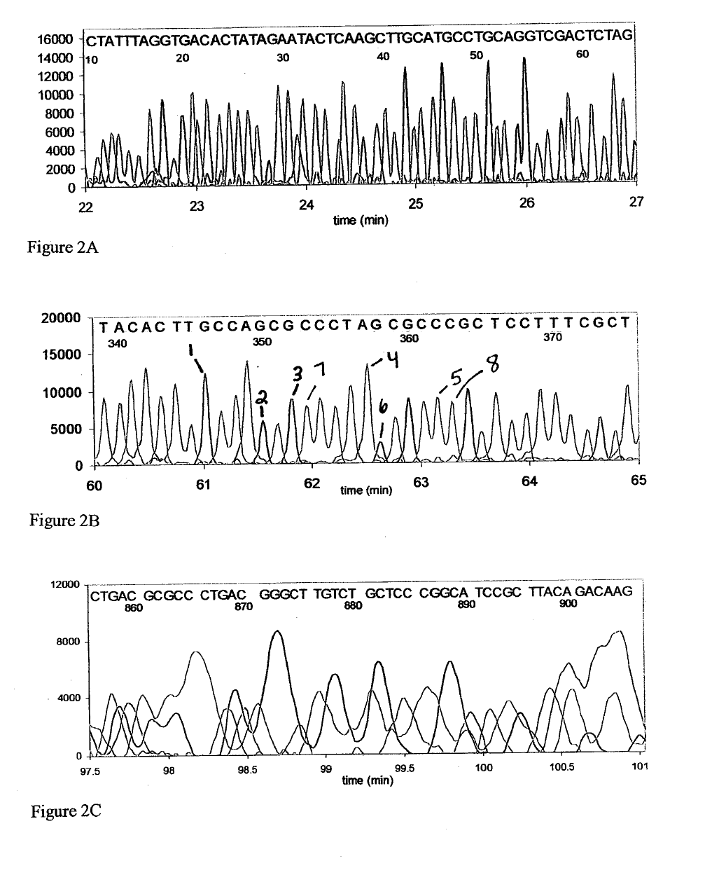

Referring to FIG. 2, fluorescence intensity-time domain data at each of 4 wavelengths derived from the separation of nucleic acid fragments are shown. The fluorescence intensity-time data is an example separations data. The observed fluorescence intensities are represented by data points, which are plotted according to the time of acquisition, τ. Hereinafter, the term “frame” is used to denote a data point. The frame acquisition time, Δτ, i.e., the time between each data point was 0.75 seconds. The term “frame number” represents the numerical position of a particular data point relative to other data points in the data. The ith frame number, N_{Fi} is given by the ratio of the time of acquisition and the frame acquisition time τᵢ/Δτ. For example, a data point acquired at a time of acquisition of 63 minutes corresponds to a frame number of 5040.

For clarity, the data in FIG. 2 only show separations data from various portions of a run, as indicated by the by the time indices along the x-axis. Features in the data, such as peaks, indicate the presence of fragments. Each color in FIG. 2 represents fluorescence from a different tag. Accordingly, peaks indicating the presence of fragments that terminate with different bases are distinguishable from one another by the wavelength of the fluorescence emitted by the tag. In addition to intensity-time domain data, the present invention is also applicable to intensity-distance data, wherein the separation of a sample is presented and/or acquired as a function of distance along a separation dimension.

110 Data Conditioning. In certain situations, the raw data must be conditioned, such as by data smoothing, baseline subtraction, or by using deconvolution techniques to identify and locate overlapped peaks. Suitable data conditioning techniques, such as those discussed below, are disclosed in U.S. application Ser. No. 09/676,526, filed Oct. 2, 2000, titled Electrophoretic Analysis System Having in-situ Calibration, which application is hereby incorporated to the extent necessary to understand the present invention.

Smoothing can be accomplished by using, for example, a Savitzky-Golay convoluting filter to improve the signal to noise ratio. Optimal properties of the filter, such as the width and order, can be determined by a user of the present invention on the basis of the signal to noise ratio of the data and the widths of peaks in the data.

Baseline subtraction can be performed to eliminate baseline drift. Typically, minima are identified in successive local sections of data, e.g., every 300 data points. Two or more minima in adjacent sections are connected, such as by a straight line or a polynomial fit to the minima. The values along the line connecting the minima are then subtracted from the intervening raw data. The new values after the baseline subtraction and smoothing are stored for further processing. The order of data smoothing and baseline subtraction can be reversed.

Overlapped peaks within the separations data can be identified and resolved using peak-fitting techniques. In most electrophoresis separations, the earlier-detected peaks are narrower than the later-detected, slower moving peaks. Within a given local section of data, however, peaks due to the presence of a single fragment have similar widths. Moreover, adjacent peaks rarely overlap exactly. Rather, the overlapped peaks a generally offset from one another. Accordingly, peaks due to the presence of multiple fragments tend to be wider than the single fragment peaks. Once a region of data containing overlapped peaks is identified, the underlying peaks can be resolved by fitting a model of the data to the observed data. Typically, the peak fitting model includes parameters that describe the amplitude, position, and width of each underlying peak.

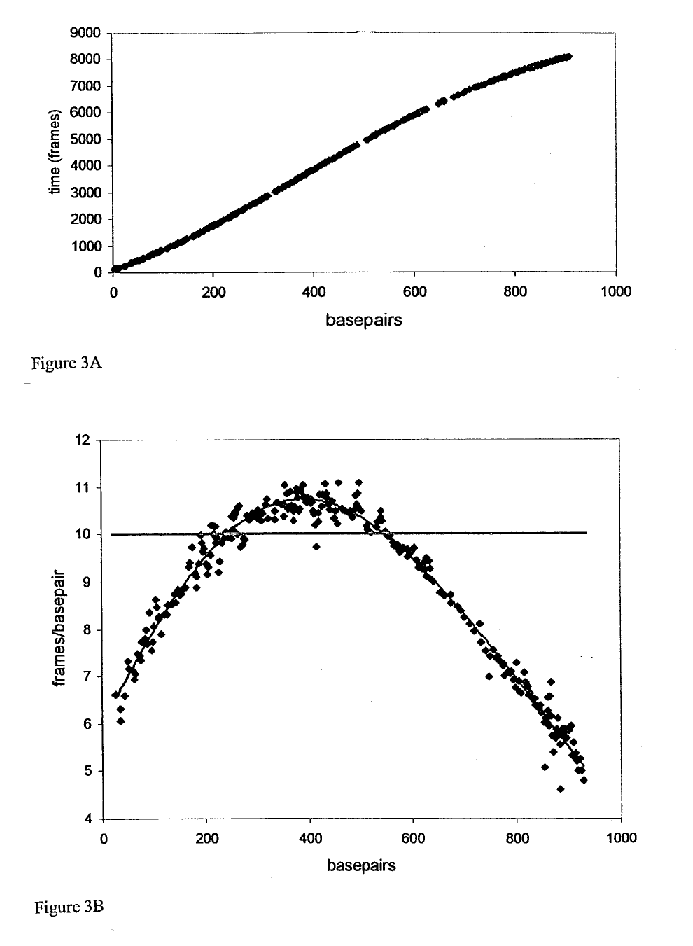

120 Determine the spacing between features. When a sieving matrix is used for an electrophoretic separation, longer DNA fragments tend to migrate more slowly than shorter fragments. The increase in migration time with fragment size, however, is generally non-linear. For example, as seen in FIG. 3A, a plot of the migration time (given in frame number) versus the number of base pairs for fragments terminating with guanine exhibits non-linear variations. A plot of migration time versus base pair derived from features in the data that indicate the presence of fragments terminating in any one of the other bases would also exhibit non-linearities similar to those in FIG. 3A.

Nonlinearities are also manifested in the rate at which successive peaks are detected. The peak detection rate is the inverse of the time or distance separating peaks in the data. For example, the migration time spacing between peaks indicating relatively smaller DNA fragments, which appear from 22 to 27 minutes in FIG. 2A, and between peaks indicating relatively larger fragments, which appear from 97.5 to 101 minutes in FIG. 2C, is smaller than the spacing between intermediately sized peaks, which appear from 60 to 65 minutes in FIG. 2B.

The spacing in time or distance between a first and second member of a pair of peaks can be determined from the relative positions of the first and second peaks. Suitable methods for determining the position of a peak are discussed in the U.S. application Ser. No. 09/676,526, which was discussed above and is incorporated herein to the extent necessary to understand the present invention.

Referring to FIG. 2B, an example of using peak maxima to determine the positions and spacing of a pair of peaks is shown. A peak 1 has a maximum at a migration time of about 61 minutes and a peak 2 has a maximum at a migration time of about 61.5 minutes. Accordingly, the spacing in time between peaks 1 and 2 is about 32 seconds or 42.5 frames. Similarly, the spacing between peak 2 and a peak 3, which has a maximum at a migration time of about 61.8 minutes, is about 17 seconds or 22.5 frames.

The spacing in time or distance between the first and second members of a pair of features is preferably normalized by a value determined on the basis of features within the data. Preferably, the normalization is determined on the basis of the number of other features, preferably peaks, appearing in the data between the first and second members of the pair. Accordingly, the spacing between peaks is preferably expressed in terms of the migration time or number of frames per feature. Because each feature corresponds to the presence of a nucleic acid, the spacing may be equivalently be described in terms of the migration time or number of frames per basepair. For example, after normalization, the spacing between peaks 1 and 2 is about 42.5 frames/4 basepairs or about 10.6 frames per basepair (about 8 seconds per basepair). After normalization, the spacing between peaks 2 and 3 is about 22.5/2 base pairs or 11.3 frames per base pair (about 8.5 seconds per basepair). Thus, in accordance with the present invention, spacings between the first and second members of respective pairs of peaks may be normalized by a respective number N where, for each pair of peaks, N is determined from the number of other peaks intermediate the first and second members.

Peaks 1-3 indicate the presence of fragments terminating with guanine, whereas the intervening peaks indicate the presence of fragments that terminate with another nucleic acid. The present invention is, however, equally adaptable to spacing data determined from the first and second members of pairs of features indicating the presence of fragments terminating with bases other than guanine. Additionally, it is not required that the first and second members of each pair indicate the presence of peaks terminating with the same base.

FIG. 3B shows a plot of the normalized spacing between the first and second members of pairs of peaks where each peak indicates the presence of a fragment that terminate with guanine. The normalized peak spacing in FIG. 3B is expressed in frames/basepair versus the base number. The plot ordinate corresponds generally to the derivative of the data in FIG. 3A. The slope of the curve reaches a maximum at around 350-400 bases and is somewhat smaller for smaller base numbers of around 100 bases and for larger base numbers around 800 bases. Thus, the average migration time spacing (peak spacing) between successive fragments terminating in guanine maximizes for fragments comprising about 350-400 bases.

A moving average approach may also be used to determine normalized spacings for separations data. For the ith peak, the moving average approach determines a normalized spacing based on a migration time difference from a pair of peaks spaced apart from the ith peak. For example, a normalized spacing at a peak 4 may be determined from the migration time difference between peak 3 and a peak 5 divided by a number N determined from the number of peaks appearing between peaks 3 and 5. Here, peak 4 may be termed the “central peak.” The migration time difference between peaks 3 and 5 is about 81 seconds or 108 frames. There are 9 peaks that appear between peaks 3 and 5. The number N is preferably given by 1 more than the number of intermediate peaks, although the number of intermediate peaks may also be used. The preferred normalized spacing, therefore, is about 8.1 seconds per basepair or about 10.8 frames per basepair. Once the normalized spacing for peak 4 is calculated, a similar normalized spacing may then calculated for a new central peak, a peak 6, but using peaks 7 and 8 as the spaced apart peaks. The spaced apart peaks may be separated by a number Np peaks from the central peak, where Np is an integer ranging from 0 to about 75.

The moving average approach, like the approach described to obtain the data in FIG. 3b, is an example of calculating a normalized spacing between first and second members of each of a plurality of pairs of peaks. A spacing in time or distance between the first and second members of each pair of peaks is determined. The spacing is normalized on the basis of the number of peaks intermediate the first and second members.

130 Fit a mapping function to the feature spacing data: A mapping function is fit to the normalized feature spacing data, such as the data shown in FIG. 3b, and used to generate a transformed migration dimension on the basis of the fit. In the transformed migration dimension, the normalized spacing between features is essentially constant except for mobility differences due, for example, to the presence of different tags. The mapping function used to fit the normalized feature spacing data preferably comprises at least one of a quadratic term in basepair number and an exponential term in basepair number.

Referring FIG. 3B, for example, the solid curve shows the best fit of

$$\frac{ⅆ {N}_{Fi}}{ⅆ x} = c + a [ X exp ( \frac{( 1 - {X}^{2} )}{2} ) - \frac{{( 1 - X )}^{2}}{4} ] \qquad ( 1 )$$

to the normalized spacing data, where X=(x−b)/380, x is basepair number, and a, b, and c are fitting constants. The fit in FIG. 3b is an example of a fit of a mapping function to the spacings between peaks. Thus, all of the spacings of the separations data may be fit with three fitting constants. In this example, the best fit constants were a=4.38, b=−30, and c=6.5.

The integral of the mapping function used to fit the feature spacing data describes generally the migration time-basepair data or, if the data are expressed in terms of migration distance, the migration distance-basepair data. For example, the integral of equation 1 is the frame number expressed as a function of basepairs, x:

$$\begin{matrix}{N}_{Fi} = {\int }_{0}^{x} \frac{ⅆ f}{ⅆ x} ⅆ x \\ = d + 380 a exp ( \frac{1 - {( b / 380 )}^{2}}{2} ) + cx + \\ \frac{380 a}{12} {( 1 - X )}^{3} - 380 a exp ( \frac{1 - {X}^{2}}{2} )\end{matrix} \qquad ( 2 )$$

where d is an offset determined by the starting point of the DNA data. Although it is possible to obtain values for the constants a, b, and c, from a fit of Equation 2 to the data shown in FIG. 3A, this fitting process generally converges more slowly than the fit of Equation 1 to the data shown in FIG. 3B. However, mapping function constants determined on the basis of a fit of the mapping function to the migration time-basepair data or migration distance-basepair data are suitable for use with the present invention. As an alternative to fitting a mapping function to the feature spacing data, a mapping function can be fit to the inverse of the feature spacing data, i.e., the rate at which successive features appear in the data.

140 Transformation of the data. Recall that the separation data of FIG. 2 include non-linear variations dependent upon at least the number of basepairs in each detected fragment and upon the particular tag associated with each fragment. Equations 1 and 2 are mapping functions that may be used to map the non-linear separation data into a space in which a plot of migration time or distance versus fragment size (basepairs) will be substantially linear for at least one of the separated species. Because the transformation substantially corrects for non-linearities generally associated with differences in fragment size, errors arising from mobility shifts between similarly sized fragments with different fluorescent tags are more readily discerned and corrected in the transformed data space. As discussed above, migration time versus basepair plots for fragments terminating with bases other than guanine are similar in form to FIG. 3A. Thus, although the fitting constants in this non-limiting example were derived from spacings between fragments terminating with guanine, similar results are obtained from fragments terminating with bases other than guanine and the present invention is not limited to mapping functions fit to guanine features.

A mapping function may be solved to predict a basepair number, xᵢ, that corresponds to the ith frame number, N_{Fi}. For example, using the fitting constants determined from the fit of equation 1 to the feature spacing data, equation 2 predicts a basepair number of about 357, for peak 1, which appears at a migration time of 61 minutes or frame number 4880. Taken together, the xᵢ can be used to generate a transformed migration time dimension or, if the separations data were acquired or expressed as a function of distance, a transformed migration distance dimension for the separated species. In the transformed space, the above-mentioned nonlinear migration time variations associated with fragment sizes are preferably substantially eliminated. Preferably, substantially the only remaining systematic variation in the peak spacing of the transformed data arises from mobility differences between fragments having different tags. By substantially eliminating non-linear variations in the data not arising from mobility differences between differentially tagged fragments, the present invention allows for accurate global correction of errors arising from the mobility differences.

150 Transformation to data having a constant spacing. In a preferred embodiment, a transformed frame number, N′_{Fi}, is generated for each frame number N_{Fi} by using the mapping function to map the time or distance corresponding to each peak onto a transformed time or distance. Preferably, the transformed frame number is directly proportional to the predicted base pair number xᵢ: N′_{Fi}=offset+k*xᵢ, where k is a predetermined constant. In the present example, k is 10 and the solid straight line in FIG. 3B represents the converted space, 10 frames/base. The constant k can be assigned any other value besides 10. The offset relates to the start of the DNA separations data, as discussed above.

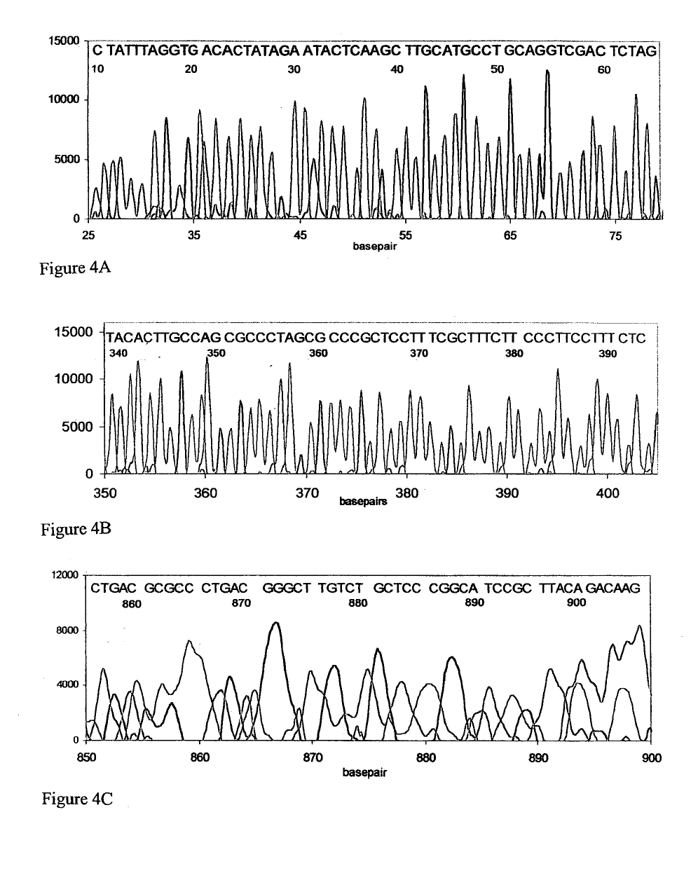

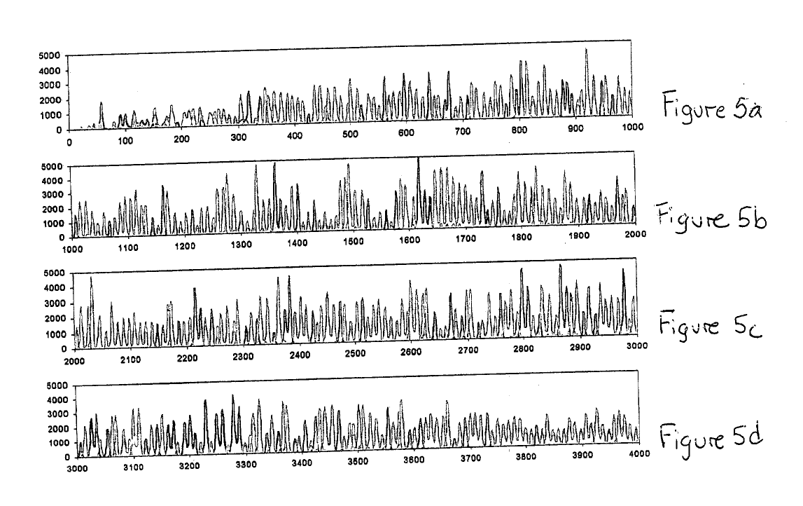

After transformation, the separations data may be represented in a data space having a nominal spacing of k frames between successive bases. For example, FIGS. 4A, 4B and 4C plot the data along the transformed abscissa of 10 frames/base or 7.5 seconds/base. The spacings between successive fragments terminating in guanine are integer multiples of the constant k so that migration time non-linearities for fragments terminating in guanine are essentially absent from the transformed data. For fragments terminating with bases other than guanine, deviations from a base spacing of k frames are indicative, as discussed below, of non-linearities not solely associated with fragment size.

Although the transformed abscissa is labeled in terms of basepairs, the transformed values along the abscissa do not correspond directly with the actual fragment size of each peak. The physically meaningful or actual fragment size is shown along the upper portion of each plot in FIGS. 4A-4C. For example, as discussed above, peak 1 appears at a transformed xi of about 357 in FIG. 4B. The actual fragment size corresponding to peak 1 is 345 basepairs as determined from the upper portion of the plot in FIG. 4B.

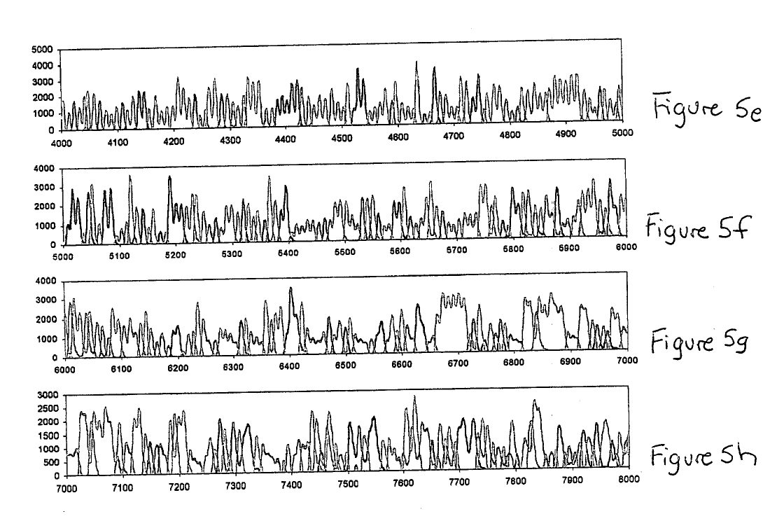

FIG. 5 shows a global overlook of the data after transformation. With the exception of the deviations discussed below, the transformed data space is substantially uniform regarding the base number or basepair. The data quality score are significantly high. Missing peaks and overcall peaks are easily identified. Because the method can be performed with optimizations of only three parameters, the method is rapid and allows automatically adjustment by computer.

The values along the transformed abscissa are generally not integers. For example, the ith value, xᵢ, may be a non-integer, such as 345.6789012. In this case, the new frame number N′_{Fi} would be 3456.78901, which does not correspond directly with an acquired frame number N_{Fi}. To complete the transformation for such values, a spline function using a few closed data points, as known in the art, can be used to interpolate the data at integer frame numbers such as, for example, 3456, 3457.

160 Correction of mobility shifts in the transformed data. In electrophoresis based DNA sequencing, mobility shifts between fragments may cause bases to be called incorrectly. For example, a base calling error typically results when mobility differences between fluorescent tags cause a longer fragment to migrate at a higher velocity than a somewhat shorter fragment so that the longer fragment is detected prior to the shorter fragment. In the absence of a correction, the nonlinear variations in the feature detection spacing or detection rate discussed above complicate attempts to correct for mobility shifts within the data. The transformed data, however, allow the presence of mobility shifts to be determined and predicted, thereby improving the accuracy of base calling.

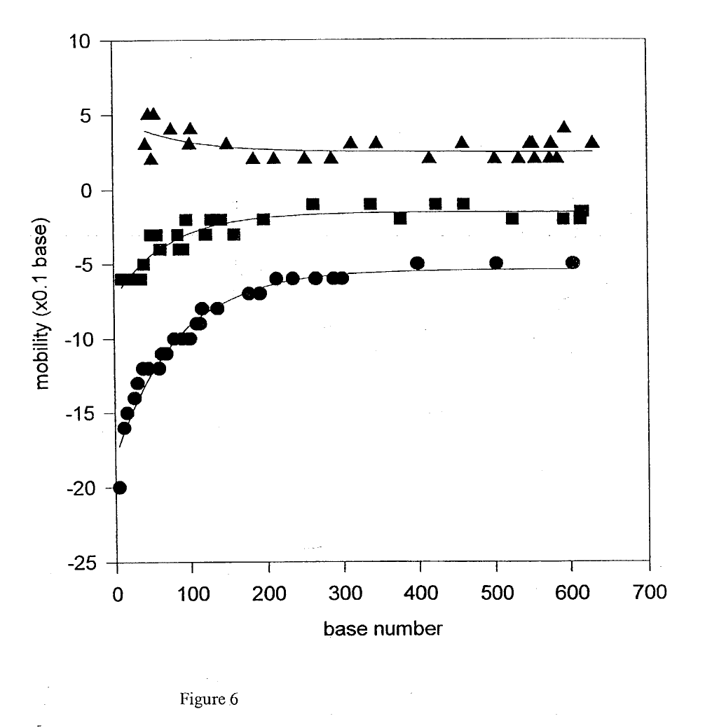

Within the transformed data, mobility shifts are manifested by deviations from k frames per base spacing. In FIG. 6, for example, the base T is chosen as a reference and peak spacings between T and the other bases are determined as a function of fragment length within the transformed data. The filled circles indicate mobility shifts for adenine to thymine, the filled squares indicate cytosine to thymine shifts, and the filled triangles indicate mobility shifts for guanine to thymine shifts.

The mobility shifts in FIG. 6 were initially determined for features at higher base numbers because the mobility shifts at higher base numbers (fragment lengths) are generally smaller than the 10 frame spacing of the transformed data. The process of determining the mobility shifts is then extended to lower base numbers where the mobility shifts exceed the 10 frame spacing of the transformed data. The process of extending the mobility shift correction is preferably assisted by fitting a mathematical function to the mobility shift data at higher base numbers and extrapolating the fit to predict mobility shifts at lower base numbers to identify shifted fragments. Preferably the fitting function comprises at least one of a constant, a polynomial term, and an exponential term. The process preferably continues until the mobility shifts have been corrected for substantially all of the transformed data.

In FIG. 6, the mobility shifts (MOB) are fit to

MOB=d+f*exp(−x/e) (3)

where x is base number and d, e, f are constants. For base C, d=−1.5±0.2, e=70±13, f=−5.8±0.6. For base A, d=−5.4±0.3, e=82±8, f=−12.6±0.5. For base G, d=2.5±0.2, e=75±72, f=2.5±1.7. The constants derived from the fitting routine allow mobility shifts to be predicted and corrected for at any point within the transformed data.

The transformed data are independent of experimental conditions, such as voltage change, capillary length, gel concentration, etc. Therefore, if separations data are obtained from samples in each of many separation lanes, the same mobility shift parameters can be applied to all of the data, and the same data processing parameters can be used for all of the capillaries. Separations data obtained from a plurality of separation lanes and transformed according to the invention may be compared to determine differences between the nucleic acid samples, such a missing base or the presence of a mutation.

The method of the invention allows the readable length of a series of bases to be extended. The readable length may be extended by at least about 100 bases. Performance of the present method exceeds that obtained by other DNA software packages, such as the DNA software package Phred.

When the base spacing is normalized to a constant, such as 10 frames/bases, peak positions may be predicted even if the peak intensity is low and multiple peaks are located in the same position. For example, a fast Fourier transform (FFT) method may be used to predict the base position in each local section containing a few hundred data points. Such a base prediction method significantly improves base calling accuracy. Suitable methods for base calling is disclosed by B. Ewing, et al. “Basecalling of Automated Sequence Using Phred (I), Accuracy Assesment”, Genome Research, 175-185, 1998 and B. Ewing, et al. “Basecalling of Automated Sequence Trace Using Phred (Ii), Error Probability”, Genome Research, 186-194, 1998.

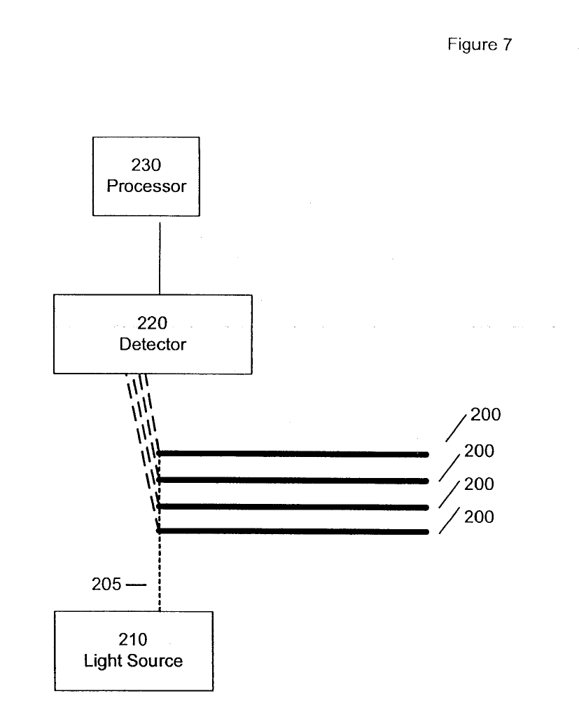

Referring to FIG. 7, an apparatus of the invention preferably includes at least one or more separation volumes, such as capillaries 200, a detector 220, and a processor 230. A light source 210 emits light 205, which is directed toward a detection region of each capillary 200. Electromagnetic radiation emitted by excited species is detected by detector 220 to obtain separations data. Processor 230 is preferably configured to transform the data based upon the spacing between features in the data to obtain transformed data having a substantially constant spacing between features. Processor 230 is preferably configured to correct mobility shifts within the data, as discussed above.

As discussed above, the data discussed herein could also be represented in terms of distance, such as a migration distance, wherein the peak spacings would be reported as a function of distance along the separation axis rather than a function of migration time. Thus, the present invention is adaptable to such an acquisition or presentation of the data.

While the above invention has been described with reference to certain preferred embodiments, it should be kept in mind that the scope of the present invention is not limited to these. One skilled in the art may find variations of these preferred embodiments which, nevertheless, fall within the spirit of the present invention, whose scope is defined by the claims set forth below.

What is claimed is:

1. A method of processing separations data, comprising:
providing first data derived from a separation of a plurality of first nucleic acid fragments, the first data comprising first features, each first feature indicating the presence of a first nucleic acid fragment terminating in one of a plurality of different bases; and
transforming the first data based upon first spacings between the first features to obtain transformed data having first transformed spacings between first features, wherein first transformed spacings between successive first features corresponding to first nucleic acid fragments terminating with one of the bases are integer multiples of a constant k.

2. The method of claim 1, wherein the first features are first peaks.

3. The method of claim 2, wherein the spacing in time or distance between first and second members of respective pairs of first peaks is normalized by a number equal to one more than the number of other peaks between the first and second members.

4. The method of claim 2, wherein transforming the data comprises fitting the first spacings between peaks to a mapping function.

5. The method of claim 4, wherein the mapping function comprises at least one of an exponential term and a quadratic term.

6. The method of claim 4, wherein transforming the data comprises using the mapping function to map the time or distance corresponding to each first peak onto a transformed time or distance.

7. The method of claim 6, further comprising determining a plurality of mobility differences in the first transformed data.

8. The method of claim 7, further comprising fitting the mobility differences to a fitting function comprising an exponential term.

9. The method of claim 8, further comprising identifying a length of one of the first nucleic acid fragments on the basis of the fit of the fitting function to the mobility differences.

10. The method of claim 2, further comprising the steps of
providing second data derived from a second separation of a plurality of nucleic acids, the second data comprising second peaks, each second peak indicating the presence of a second nucleic acid fragment terminating in one of a plurality of different bases;
transforming the second data based upon spacings between the second peaks to obtain second transformed data having second transformed spacings between peaks, wherein second transformed spacings between successive peaks corresponding to second nucleic acid fragments terminating with one of the bases are integer multiples of the constant k; and
comparing the first and second transformed data to determine a difference between the transformed data.

11. An electrophoresis separation apparatus having at least one separation lane, a detector, and a processor, wherein the processor is configured to:
obtain intensity-time data from the electrophoretic separation, the intensity-time data comprising peaks, each peak associated with the presence of a nucleic acid fragment terminating in one of a plurality of different bases; and
transform the data based upon first spacings between the peaks to obtain transformed data having transformed spacings between peaks, wherein transformed spacings between successive peaks corresponding to nucleic acid fragments terminating with one of the bases are integer multiples of a constant k.

12. The apparatus of claim 11, wherein the electrophoretic separation apparatus comprises a plurality of separation lanes and the apparatus is adapted to obtain a plurality of intensity-time data associated with simultaneous separation of a plurality of nucleic acid samples.

13. The apparatus of claim 11, wherein the processor is configured to determine a spacing in time or distance spacing between peaks and to normalize the spacing in time or distance between members of respective pairs of peaks.

14. The apparatus of claim 13, wherein the processor is configured to fit the normalized spacing between features to a mapping function.

15. The apparatus of claim 14, wherein the processor is configured to map the time or distance corresponding to each feature onto a transformed time or distance.

16. The apparatus of claim 15, wherein the processor is further configured to determine a plurality of mobility differences in the transformed data.

17. The apparatus of claim 16, wherein the processor is configured to fit the mobility differences to a fitting function and determine a length of at least one of the nucleic acid fragments.

18. A method of processing data derived from a separation of a nucleic acid sample, comprising:
obtaining separations data, the data comprising peaks indicating the presence of different nucleic acid fragments in the sample;
calculating a normalized spacing between first and second members of each of a plurality of pairs of peaks by determining a spacing in time or distance between the first and second members of each pair and normalizing the spacing based on a number of peaks intermediate the first and second members; and
transforming the separations data based upon the normalized spacings to obtain transformed data having a substantially constant spacing between adjacent peaks.

19. An electrophoresis separation apparatus having a plurality of separation lanes for separating a respective nucleic acid sample, a detector, and a processor, wherein the processor is configured to:
obtain respective intensity-time data from each separation lane, the intensity-time data comprising peaks, each peak associated with the presence of a nucleic acid fragment terminating in one of a plurality of different bases;
transform respective intensity data based upon spacings between the peaks to obtain respective transformed data having transformed spacings between peaks, wherein transformed spacings between successive peaks corresponding to nucleic acid fragments terminating with one of the bases are integer multiples of a constant k; and
comparing the transformed data from different separation lanes to determine differences between the nucleic acid samples.

20. A method of processing separations data, comprising:
providing separations data from the separation of a nucleic acid sample comprising a plurality of nucleic acid fragments, the separations data comprising peaks, each peak indicative of the presence of a nucleic acid fragment terminating in one of a plurality of different bases; and
transforming the separations data based upon spacings between first and second members of respective pairs of the peaks, wherein the spacing between the first and second members of each pair is normalized by a respective number N determined from the number of peaks intermediate the first and second members.

21. The method of claim 20, further comprising
providing second separations data from a second separation of a second nucleic acid sample comprising a plurality of second nucleic acid fragments, the second separations data comprising second peaks, each second peak indicative of the presence of a second nucleic acid fragment terminating in one of a plurality of different bases;
transforming the second separations data based upon second spacings between first and second members of respective pairs of the second peaks, wherein the second spacing between the first and second members of each pair is normalized by on the basis of the number of peaks intermediate the first and second members; and
comparing the separations data and the second separations data to determine a difference between the nucleic acid sample and the second nucleic acid sample.
